(19) United States
(12) Patent Application Publication
(10) Pub. No.: US 2011/0009340 A1
(43) Pub. Date: Jan. 13, 2011

(54) COMBINATION OF AN IRON CHELATOR AND AN IMMUNOSUPPRESSANT AND USE THEREOF

(75) Inventor: Oliver Leismann, Nuernberg (DE)

Correspondence Address: NOVARTIS;CORPORATE INTELLECTUAL PROPERTY, ONE HEALTH PLAZA 101/2, EAST HANOVER, NJ, 07936-1080, US

(73) Assignee: NOVARTIS AG, BASEL (CH)

(21) Appl. No.: 12/866,932

(22) PCT Filed: Feb. 18, 2009
(86) PCT No.: PCT/EP09/51901

§ 371 Date: Aug. 10, 2010

(30) Foreign Application Priority Data
Feb. 19, 2008 (EP) 08151642.9

(51) Int. Cl. A61K 38/13; A61P 7/06; A61P 37/06

(52) U.S. Cl. 514/20.5

(57) ABSTRACT

The invention relates to a combination comprising an iron chelator and an immunosuppressant, to the use of such combination for the improvement of immunosuppression, e.g. in hematopoietic stem cell transplantation.

The invention provides a combination comprising an iron chelator and an immunosuppressant. The invention provides for the use of said combination, e.g. in transplantation, e.g. in allogenic stem cell transplantation.

Patients with hematologic malignancies who undergo an allogeneic hematopoietic cell transplantation (HCT) often receive multiple blood transfusions. Only a few publications exist addressing the problem of secondary hemochromatosis due to multiple blood transfusions in patients after HCT (Oguchi, T. 1995, Mahendra, P. 1996). Phlebotomy to remove excess iron is the standard treatment in patients with primary (hereditary) hemochromatosis. However, in patients with hematologic malignancies requiring multiple blood transfusions, phlebotomy prior to HCT is often not possible due to anemia. Thus, there is a need for an improved management of transplanted patients, e.g. HCT patients.

The invention provides a method to improve transplantation, e.g. stem cell transplantation, which method comprises co-administering to a mammal, especially a human, in need of such treatment, a combination of an iron chelator and an immunosuppressant. The iron chelator and the immunosuppressant are preferably co-administered in an amount such that the combination has a desired therapeutic effect.

By “immunosuppressive agent” is meant a cyclosporine, or ascomycine or their immunosuppressive analogs or derivatives, e.g. cyclosporin A, cyclosporin G, FK-506, ABT-281, ASM 981; an mTOR inhibitor, e.g. rapamycin, 40-O-(2-hydroxy)ethyl-rapamycin.

Cyclosporin A (SANDIMUN®, NEORAL®) is a well-known immunosuppressive agent and particular used for the prevention of transplant rejection, including liver transplant rejection.

Cyclosporin A and cyclosporin A derivatives are known and described, e.g., in U.S. Pat. No. 4,117,118 or European Patent No. EP 0 539 319. Cyclosporin A derivatives include cyclosporine A prodrugs as described, e.g., in J Peptide Res, Vol. 63, pp. 147-154 (2004). Cyclosporin A formulations described, e.g., in EP 0 539 319 or U.S. Pat. No. 5,234,625 form a microemulsion in an aqueous environment, particularly as commercially-available under the tradename NEORAL®.

The pharmaceutical formulations of cyclosporin A or the derivative thereof are preferably a “microemulsion pre-concentrate” as indicated above, the individual components or ingredients of which are pharmaceutically acceptable, e.g., where oral administration is foreseen for oral use.

In addition to the cyclosporin active ingredient, such “microemulsion pre-concentrate” compositions generally comprise:
1) a hydrophilic phase;
2) a lipophilic phase; and
3) a surfactant.

The cyclosporin is carried in the lipophilic phase. Suitably both the hydrophilic and lipophilic phases may serve as carrier medium.

“Microemulsion pre-concentrates” of the invention are of a type providing oil-in-water (o/w) microemulsions. As will be appreciated, however, microemulsion pre-concentrate compositions may contain minor quantities of water or otherwise exhibit fine structural features characteristic of microemulsions, e.g., of o/w or water-in-oil (w/o) type. The term “microemulsion pre-concentrate”, as used herein, is accordingly to be understood as embracing such possibilities.

Microemulsions obtained on contacting the “microemulsion pre-concentrate” compositions of the invention with water or other aqueous medium exhibit thermodynamic stability, that is they will remain stable at ambient temperatures, e.g., without clouding or regular emulsion size droplet formation or precipitation, over prolonged periods of time. While the upper limit of dilution with water is not critical, a dilution of 1:1, preferably 1:5 parts per weight (“microemulsion pre-concentrate”:H₂O) or more will generally be appropriate. Preferably, on contacting with water, the “microemulsion pre-concentrate” compositions provide microemulsions having an average particle size of less than about 1,500 angstroms (Å), more preferably of less than about 1,000 Å or 1,100 Å, e.g., down to about 150 Å or 200 Å.

The term “mTOR inhibitor” as used herein includes, but is not limited to rapamycin (sirolimus) or a derivative thereof. Rapamycin is a known macrolide antibiotic produced by Streptomyces hygroscopicus. Suitable derivatives of rapamycin include e.g. compounds of formula A

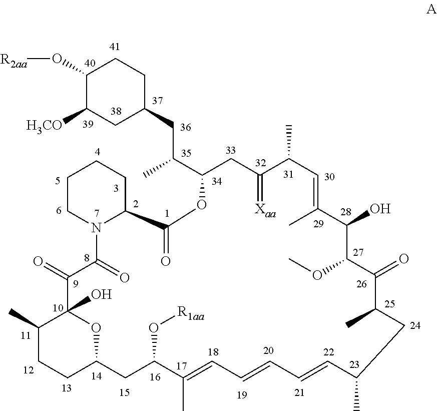

wherein
R1aa is CH3 or C3-6 alkynyl,
R2aa is H or —CH2—CH2—OH, 3-hydroxy-2-(hydroxymethyl)-2-methyl-propanoyl or tetrazolyl,
and

### Xaa is ═O, (H,H) or (H,OH)

provided that R2aa is other than H when Xaa is ═O and R1aa is CH3.
or a prodrug thereof when R2aa is —CH2—CH2—OH, e.g. a physiologically hydrolysable ether thereof.

Compounds of formula A are disclosed e.g. in WO 94/09010, WO 95/16691, WO 96/41807, U.S. Pat. No. 5,362,718 or WO 99/15530 which are incorporated herein by reference. They may be prepared as disclosed or by analogy to the procedures described in these references. Preferred rapamycin derivatives are 32-deoxorapamycin, 16-pent-2-ynyloxy-32-deoxorapamycin, 16-pent-2-ynyloxy-32(S)-dihydro-rapamycin, 16-pent-2-ynyloxy-32(S)-dihydro-40-O-(2-hydroxyethyl)-rapamycin and, more preferably, 40-O-(2-hydroxyethyl)rapamycin. Further examples of rapamycin derivatives include e.g. CCI779 or 40-[3-hydroxy-2-(hydroxymethyl)-2-methylpropanoate]-rapamycin or a pharmaceutically acceptable salt thereof, as disclosed in U.S. Pat. No. 5,362,718, ABT578 or 40-(tetrazolyl)-rapamycin, particularly 40-epi-(tetrazolyl)-rapamycin, e.g. as disclosed in WO 99/15530, or rapalogs as disclosed e.g. in WO 98/02441 and WO01/14387, e.g. AP23573 or TAFA-93.

The iron chelators to which the present invention applies are any of those having pharmaceutical utility, e.g. as therapeutic agents for the chelation of iron, e.g. in patient in need for iron chelation.

Iron chelators according to the present invention are for example, deferoxamine, deferasirox, deferiprone, L1NAll, and deferitrin.

Desferal, deferoxamine mesylate USP, is an iron-chelating agent, available in vials for intramuscular, subcutaneous, and intravenous administration. Desferal is supplied as vials containing 500 mg and 2 g of deferoxamine mesylate USP in sterile, lyophilized form. Deferoxamine mesylate is N-[5-[3-[(5-aminopentyl)hydroxycarbamoyl]propionamido]pentyl]-3-[[5-(N-hydroxyacetamido)pentyl]carbamoyl]propionohydroxamic acid monomethanesulfonate (salt).

Deferoxamine mesylate USP is a white to off-white powder. It is freely soluble in water and slightly soluble in methanol. Its molecular weight is 656.79, and its structural formula is:

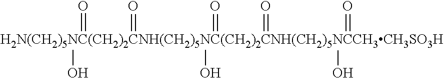

An iron chelator according to the present invention can be a 3,5-diphenyl-1,2,4-triazole derivative of formula (I)

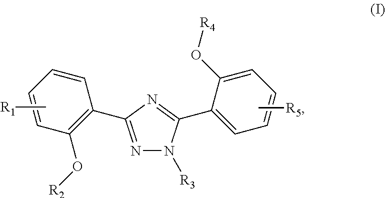

in which
R₁ and R₅, simultaneously or independently of one another, are hydrogen, halogen, hydroxyl, lower alkyl, halo-lower alkyl, lower alkoxy, halo-lower alkoxy, carboxyl, carbamoyl, N-lower alkylcarbamoyl, N,N-di-lower alkylcarbamoyl or nitrile;
R₂ and R₄, simultaneously or independently of one another, are hydrogen, unsubstituted or substituted lower alkanoyl or aroyl, or a radical which can be removed under physiological conditions;
R₃ is hydrogen, lower alkyl, hydroxy-lower alkyl, halo-lower alkyl, carboxy-lower alkyl, lower alkoxycarbonyl-lower alkyl, R₆R₇N—C(O)-lower alkyl, unsubstituted or substituted aryl or aryl-lower alkyl, or unsubstituted or substituted heteroaryl or heteroaralkyl;
R₆ and R₇, simultaneously or independently, of one another are hydrogen, lower alkyl, hydroxy-lower alkyl, alkoxy-lower alkyl, hydroxyalkoxy-lower alkyl, amino-lower alkyl, N-lower alkylamino-lower alkyl, N,N-di-lower alkylamino-lower alkyl, N-(hydroxy-lower alkyl)amino-lower alkyl, N,N-di(hydroxy-lower alkyl)amino-lower alkyl or, together with the nitrogen atom to which they are bonded, form an azaalicyclic ring;
   or a salt thereof.

Preferably, a 3,5-diphenyl-1,2,4-triazole derivative of the present invention is 4-[3,5-bis(2-hydroxyphenyl)-[1,2,4]trizol-1-yl]benzoic acid, herein after referred as deferasirox, or a pharmaceutically acceptable salt thereof. Deferasirox is described in, for example EP0914118, and in U.S. Pat. No. 6,465,504 B1. Pharmaceutical preparation comprising deferasirox are disclosed, e.g. in the following International Patent Application WO2004/035026, Deferasirox can be administered according to the manufacturer's instructions.

The term “iron chelator” according to the present invention also encompasses the following listed compounds:

Deferitrin or GT56-252 having the following formula 4,5-dihydro-2-92,4-dihydroxyphenyl)-4-methylthiazole-4(S)-carboxylic acid.

Deferiprone is 3-hydroxy-1,2-dimethylpyridin-4(1H)-one.

L1NAll is the second generation deferiprone (L1) analogue that has the following formula 1-allyl-2-methyl-3-hydroxypyrid-4-one.

The term “co-administration” of a combination of an iron chelator, e.g. deferasirox, or deferoxamine or deferiprone or deferitrin or L1NAll and an immunosuppressant, e.g. cyclosporine A means that the components can be administered together as a pharmaceutical composition or as part of the same, unitary dosage form. Co-administration also includes administering an iron chelator, e.g. deferasirox, or deferoxamine or deferiprone or deferitrin and an immunosuppressant separately but as part of the same therapeutic regimen. The components, if administered separately, need not necessarily be administered at essentially the same time, although they can if so desired. Thus, co-administration includes, e.g., administering an iron chelator e.g. deferasirox, or deferoxamine or deferiprone or deferitrin, plus an immunosuppressant as separate dosages or dosage forms, but at the same time. Co-administration also includes separate administration at different times and in any order, e.g. an iron chelator can be administered prior to an immunosuppressant or vice versa. The administration of an iron chelator can also occur at different time points before or after the HCT, e.g. 3 to 6 months after the HCT.

The present invention provides a method of treating a patient having received an HCT, which method comprises administering to said patient an immunosuppressant, e.g. a cyclosporin A or a cyclosporin A derivative in association with an iron chelator.

In a further aspect, the invention relates to the use of an immunosuppressant, e.g. a cyclosporin A or a cyclosporin A derivative in the manufacture of a medicament for the prevention of graft versus host rejection in association with an iron chelator, and in yet another aspect to the use of an iron chelator in the manufacture of a medicament for the prevention of graft versus host rejection, e.g. HCT, in association with cyclosporin A or a cyclosporin A derivative.

The iron chelator and the immunosuppressant are administered in an amount such as that, for example, the immunosuppression is improved in comparison with the immunosuppressant taken alone. The iron chelator and the immunosuppressant are administered in an amount such as that, for example, the immunosuppression is improved in comparison with the immunosuppressant being combined with phlebotomy.

Further advantages resulting from the combination therapy of the invention can be
improvements such as higher overall and disease free survival rate after transplantation, e.g. HCT,
reduction in hepcidin production,
reduced acute graft versus host disease,
reduce treatment related mortality after transplantation, e.g. HCT,
reduce chronic liver disease after HCT,
creatinine levels not higher than creatinine level generated by the treatment with the immunosuppressant alone.

The combination of an iron chelator and an immunosuppressive agent can improve the absorption of said immunosuppressive agent and/or improve the immunosuppression, e.g. in transplanted patients, e.g. in HCT patients.

Preferably, a pharmaceutical composition of the present invention comprises an iron chelator, e.g. a 3,5-diphenyl-1,2,4-triazole derivative or a salt thereof, e.g. deferasirox, deferiprone, deferitrin, L1NAll, deferoxamine; in combination with an immunosuppressant, e.g. cyclosporin A, cyclosporin G, FK-506, ABT-281, ASM 981; an mTOR inhibitor, e.g. rapamycin, 40-O-(2-hydroxy)ethyl-rapamycin.

The present invention provides a pharmaceutical composition comprising a therapeutically effective amount of an iron chelator, preferably, a 3,5-diphenyl-1,2,4-triazole derivative, in combination with an immunosuppressant, e.g. cyclosporin A, cyclosporin G, FK-506, ABT-281, ASM 981; an mTOR inhibitor, e.g. rapamycin, 40-O-(2-hydroxy)ethyl-rapamycin.

Preferably, a pharmaceutical composition of the present invention comprises a iron chelator, e.g. deferasirox and an immunosuppressant, e.g. cyclosporin A,

Since the present invention has an aspect that relates to treatment with a combination of compounds which may be co-administered separately, the invention also relates to combining separate pharmaceutical compositions in kit form. The kit comprises two separate pharmaceutical compositions:
(1) a composition comprising one iron chelator, in particular, a 3,5-diphenyl-1,2,4-triazole derivative, e.g. 4-[3,5-bis(2-hydroxyphenyl)-[1,2,4]triazol-1-yl]benzoic acid plus a pharmaceutically acceptable carrier or diluent; and
(2) a composition comprising a cyclosporine, e.g. cyclosporin A.

The kit comprises a container for containing the separate compositions, such as a divided bottle or a divided foil packet, wherein each compartment contains a plurality of dosage forms (e.g., tablets) comprising (1) or (2). Alternatively, rather than separating the active ingredient-containing dosage forms, the kit may contain separate compartments each of which contains a whole dosage which in turn comprises separate dosage forms. An example of this type of kit is a blister pack wherein each individual blister contains two (or more) tablets, one (or more) tablet(s) comprising a pharmaceutical composition (1), and the second (or more) tablet(s) comprising a pharmaceutical composition (2). Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician. In the case of the instant invention a kit therefore comprises:
(1) a composition comprising an iron chelator, e.g. a 3,5-diphenyl-1,2,4-triazole derivative, and at least one pharmaceutically acceptable carrier or diluent, in a first dosage form;
(2) a composition comprising at least one cyclosporine, in a second dosage form; and
(3) a container for containing said first and second dosage forms.

The Examples herein are not a limitation of the scope of the present invention in any way.

### EXAMPLES

### Example 1

### Efficacy and Safety of Oral Deferasirox, e.g. Administered at 20 mg/kg/day in Patients Three to Six Months after Allogenic Hematopoietic Cell Transplantation in Whom Iron Overload is Present

One year, open labeled single arm multi center trial evaluating the efficacy and safety of oral deferasirox, e.g. administered in patients three to six months after allogenic hematopoietic cell transplantation in whom iron overload is present.

The effect of iron chelation using deferasirox is investigated in patients who show signs of iron overload after an allogenic stem cell transplantation. The iron overload can, for example, be due to blood transfusions.

For example 75 patients are recruited in the clinical trial. Patients shall be 3 to 6 months after allogeneic hematopoietic stem cell transplantation.

Factors related to iron stores in the participating patients such as number of blood transfusions from diagnostic, HFE genotype of the patients after HCT, hepcidin after HCT, which may influence iron body stores after HCT are monitored.

The iron chelation is assessed by comparing serum ferritin values at baseline versus 52 weeks of treatment with deferasirox. The influence of deferasirox on the absorption of cyclosporine A is monitored. The immunosuppression, e.g. by measuring cyclosporine A levels during the whole study is monitored. The allogenic hematopoietic cell transplantation is also monitored in order to determine a potential improvement in the transplantation.

The incidence of chronic graft-versus-host disease at day 365 post-transplant according to Shulman criteria is monitored.

### Study Design:

The daily dose of deferasirox is escalated in every patient during the initial study phase starting with 10 mg/kg body weight/day at day one of study treatment and reaching the daily dose of 20 mg/kg of body weight after 4 weeks. The 20 mg/kg/day is maintained during the residual 48 week treatment period or until a serum ferritin level inferior to 500 ng/ml is reached, e.g. whichever is the first, unless a dose adjustment is deemed necessary.

### Inclusion Criteria:

1. Transfusional iron overload three to six months after the hematopoietic stem cell transplantation, e.g. mean serum ferritin level superior to 1000 ng/ml, with no evidence of active inflammation
2. History of at least 20 units of red blood cells transfusions or 100 mL/kg of prepacked red blood celles (PRBCs).
3. Patients of either gender and age superior or equal to 18 years
4. Female patients who have reached menarche and who are sexually active must use double-barrier contraception, oral contraceptive plus barrier contraception or must have undergone clinically documented total hysterectomy and/or ovariectomy, or tubal ligation or be postmenopausal defined by amenorrhea for at least 12 months.

### Exclusion Criteria:

1. Non-transfusion related iron overload
2. Active malignancy
3. Known active viral hepatitis or known HIV positiveness
4. Mean levels of alanine aminotransferase (ALT)>5×ULN
5. Treatment with any iron chelator after transplantation
6. Uncontrolled systemic hypertension
7. Serum creatinine >1.5 ULN and/or serum creatinine clearance <60 ml/min
8. History of nephrotic syndrome.
9. Previous history of clinically relevant ocular or auditory toxicity related to iron chelation.
10. Systemic diseases (cardiovascular, renal, hepatic, etc.) which would prevent the patient from undergoing study treatment
11. Pregnant or breast feeding patients.

Parameters monitored on the patients are for example, clinical examination. Cyclosporin A trough levels, serum creatinine, transfusion, HFE genotype, electrocardiogram, hepcidin in urine, iron metabolism, deferasirox pharmacokinetic tests, protein in the urine, signs of chronic graft versus host disease using the Shulman criteria and protein in urine.

### Chronic GvHD May be Defined as Limited or Extensive Using the Following Criteria Defined by Shulman

### Limited Chronic GvHD

Either or both criteria must be present:
Localised skin involvement and/or
Hepatic dysfunction

### Extensive Chronic GvHD

### Either:

Generalised skin involvement

### Or

Localised skin involvement and/or hepatic dysfunction
plus
Liver histology showing chronic aggressive hepatitis,
bridging necrosis or cirrhosis
or
Involvement of eye: Schirmer's test with <5 mm
wetting,
or
Involvement of minor salivary glands or oral mucosa
demonstrated on labial biopsy specimen,
or
Involvement of any other target organ (e.g., oesophageal
abnormalities, polymyositis)

1. A combination comprising (a) an iron chelator and, (b) an immunosuppressant.

2. A combination according to claim 1, wherein the iron chelator is selected from deferoxamine, deferasirox, deferiprone, L1NAll or deferitrin or a pharmaceutically acceptable salt thereof.

3. The combination according to claim 1 wherein the immunosuppressant is selected from the group consisting of cyclosporin A, a cyclosporine A derivative, cyclosporin G, FK-506, ABT-281, ASM 981, rapamycin, and 40-O-(2-hydroxy)ethyl-rapamycin.

4. The combination according to claim 1 wherein the iron chelator is deferasirox and the immunosuppressant is cyclosporine A or cyclosporine A derivative.

5. The combination according to claim 4 wherein the immunosuppressant is cyclosporine A or cyclosporine A derivative in a microemulsion pre-concentrate.

6. A method of treatment for a patient undergoing or having received a HCT comprising administering a combination according to claim 1.

7. The combination according to claim 6 wherein the patient received a HCT from a donor having a mutant HFE gene.

8. A pharmaceutical composition comprising a combination according to claim 1.

9-10. (canceled)
